(19) United States
(12) Patent Application Publication
(10) Pub. No.: US 2017/0182058 A1
(43) Pub. Date: Jun. 29, 2017

(54) TREATMENT OF RETT SYNDROME

(71) Applicant: EMICIPI LLC, Palm Beach Gardens, FL (US)

(72) Inventors: Bonnie M. DAVIS, Palm Beach Gardens, FL (US); Joanne BERGER-SWEENEY, Hartford, CT (US)

(73) Assignee: EMICIPI LLC, Palm Beach Gardens, FL (US)

(21) Appl. No.: 15/128,746

(22) PCT Filed: Mar. 24, 2015
(86) PCT No.: PCT/US2015/022210

§ 371 Date: Sep. 23, 2016

Related U.S. Application Data
(60) Provisional application No. 61/969,908, filed on Mar. 25, 2014.

(51) Int. Cl. A61K 31/55; A61K 9/50; A61K 9/00

(52) U.S. Cl. CPC A61K 31/55; A61K 9/0053; A61K 9/5021

(57) ABSTRACT

Rett syndrome (RTT), a childhood neurological disorder that affects primarily females, may be treated by use of a galanthamine analog wherein the hydroxy group of galantamine is replaced of a carbarnate, carbonate or ester group and the methoxy group may be replaced by another alkoxy group of from two to six carbon atoms, a hydroxy group, hydrogen, an alkanoyloxy group or 2 to 10 carbon atoms, a benzoyloxy or substituted benzoyloxy group, a carbonate group of 1 to 10 carbon atoms or a carbamate group such as a mono alkyl or dialkyl or an aryl carbamate wherein the alkyl grottos or aryl groups contain from 1 to 10 carbons; and the N-methyl group may be replaced by hydrogen, alkyl of 1 to 10 carbon atoms benzyl, cyclopropylmethyl group or a substituted or unsubstituted benzoyloxy group. Galantamine mon-alkylcarbarnates are particularly useful.

### CROSS REFERENCE TO RELATED APPLICATION

The present application claims priority from provisional application 61/969,908 filed on Mar. 25, 2014, the entire contents of which are incorporated herein by reference.

### FIELD OF THE INVENTION

The present invention relates to a method of treating Rett Syndrome patients in order to reduce cognitive deficits in symptomatic patients.

### BACKGROUND OF THE INVENTION

Rett syndrome (RTT) is a childhood neurological disorder that affects primarily females. RTT has features observed commonly in many other disorders ranging from autism to Parkinson's disease, including reduced social interactions, mental retardation, reduced head growth, abnormal motor skills, emotional disturbances and abnormal respiration (Katz, Berger-Sweeney 2012). RTT is the second most prevalent cause of mental retardation in girls, with a prevalence of about 1:10,000 and, as with other neurodevelopmental disorders with associated mental retardation, is resistant to treatment.

RTT has a complex phenotype and a unique onset of symptoms. Ordinarily, Rett girls are born full term after normal pregnancies and with few reported perinatal problems. The girls develop relatively normally for the first six months of life followed by a period of regression. The girls generally present to doctors with neurologic regression, usually starting between 6 months and 2 years, with loss of acquired hand skills and spoken language and, in some cases, social withdrawal or extreme irritability that can resemble autism. After regression, there is a pseudo-stabilization stage and during this stage, characteristic features of RTT, such as repetitive hand movements (stereotypies), often appear.

RTT girls have reduced height, weight, and head growth, although a subset is overweight. The girls have various gastrointestinal problems and it is not clear if the reduced growth is a function of malnutrition or not. Abnormal recordings on electroencephalography and clinical epilepsy are also common features by age 10. Motor dysfunction, scoliosis, and respiratory problems are also common, and the latter problem can be fatal in a large number of cases. Later in life, many RTT girls exhibit motor decline, and Parkinsonian features such as rigidity and freezing become prominent. Other motor disorders include gait abnormalities, tremors, myoclonus, chorea, and severe teeth grinding. During the regression stage, autistic features appear including social withdrawal, avoidance of eye contact, indifference to visual or auditory stimuli, and sensitivity to novel situations. It is not clear whether autistic features remain prominent throughout life or are reduced as the girls aged.

There is relatively little known about cognitive functioning and information processing capacity of individuals with RTT and even less information about their cognitive potential [Berger-Sweeney 2010]. The evaluation of cognitive functions and assessment of developmental levels in children almost always depend on verbal or gesture responses, and are focused on the knowledge that the child acquires about the material world. The marked communication deficits accompanied by severe motor impairments in RTT girls make it extremely difficult to evaluate their cognitive capacity. Although there are numerous reports of severely limited cognitive and communication skills in RTT individuals, there is little indication that these skills deteriorate significantly with age, rather the development of these skills appears to be arrested around the time of onset of the regression, in other words, typically between 6 and 18 months of age. In contrast to the findings of pervasive and general intelligence impairments, there are reports that RTT girls have increased social interaction with caregivers over time suggesting that elements of social memory are intact. Though most RTT girls exhibit no or low levels of object permanency, the knowledge of objects that are no longer visible, many parents report anecdotes suggesting moderate recognition skills and reactions that suggest that the girls understand more than developmental tests indicate.

In an attempt to examine cognitive capacity and avert the severe motor and communication deficits in RTT girls, several studies have examined intentional eye gaze using ocular tracking. Fixed gazes and intentional stares are forms of communication that express particular desires. However, there is little evidence that the fixed gazes equate with an ability to learn or remember objects.

Breathing abnormalities are common in RTT. Respiratory abnormalities include periods of forceful breathing (hyperventilation), breathing pauses and abnormal cardiorespiratory coupling, and these symptoms are more severe during wakefulness than during sleep and may be exaggerated with excitement or stress. One quarter of deaths in RTT are sudden and unexpected and may relate to respiratory dysfunction.

Girls with RTT exhibit a high rate of epileptic seizures (focal, multifocal, and generalized eliptiform abnotmalities) and atypical electroencephalographic (EEG) patterns, generally emerging in the teen years.

Though the brains of RTT patients are generally smaller than normal, they do not exhibit gross neuropathological changes. Generally, neuronal or glial atrophy, degeneration, gliosis, or demyelination is not evident, suggesting that RTT not a neurodegenerative disorder. RTT brains do exhibit generally smaller total brain volume and smaller, densely packed neurons in neocortex, hippocampus, and hypothalamus. Dendritic arborizations and spine density are reduced in neocortex and hippocampus, suggesting reduced neurotransmission and connectivity in RTT brains.

A number of treatments have been tried with RTT individuals, but clinical improvements have been elusive. Growth factors and nutritional supplements have been assessed, however, without clinical success to date. The most recent trials involve insulin-like growth factor—1 (IGL1), however, positive results have not yet been reported. There is a critical need to develop effective therapies for this devastating disorder.

There has been an increased interest in Rett syndrome after the discovery of the gene found in affected individuals. Over 80% of RTT cases in girls are due to mutations in the X-linked gene encoding methyl-CpG-binding protein 2 (MeCP2). Identification of the mutant gene has also led to the discovery of MeCP2-related severe phenotypes in males. MeCP2 is a multifunctional protein, whose role as a transcriptional repressor is best studied. More recently, there is also evidence highlighting a role for MeCP2 in activation of numerous genes. MeCP2 binds to the promoter region to activate target genes. A mutation in MeCP2, as is in the case in RTT, leads to the dysregulation of a number of genes that are normally methylated and stably repressed. Recently, several other functional roles for MeCP2 have been noted and are beginning to be characterized more fully, including binding to non-promoter regions of DNA. The precise role of MeCP2 mutations in producing the clinical RTT phenotype is still unclear although it is clear that MeCP2 regulates numerous functional genes, and the list of genes likely regulated by MeCP2 is increasing each year. Brain derived neurotrophic factor (BDNF) is one gene that is regulated by MeCP2.

Currently, a variety of mouse models with altered MeCP2 expression exist, all of which reprise key symptoms of the RTT phenotype. The behavioral phenotypes of mutant male mice, which lack functional MeCP2 protein, are surprisingly similar for those features that have been characterized including stereotypies, motor, respiratory, and social and cognitive deficits, and reduced body weight and brain size [Katz and Berger-Sweeney 2012]. Female mutant mice, which more closely resemble the genetics of RTT exhibit variable severity of symptoms and symptoms are milder with a longer time to onset than those in males. Thus, the mutant males are a more popular model that is considered to replicate better the most severely affected RTT girls, but clinical efficacy will only be achieved when mutant females are included.

One of the most consistent neurochemical abnormalities described in girls with RTT is the loss of cholinergic neurons in the basal forebrain and reduced cholinergic functioning. Acetylcholine (ACh) is an essential neurotransmitter in adulthood controlling selective attention, learning and working memory. ACh is also an essential neuromodulator during critical time windows in cortical development to regulate formation of neuronal networks necessary for cognition. The major source of acetylcholine conies from neurons that reside in the basal forebrain and project to neocortex and hippocampus. Cholinergic neurotransmission can be enhanced through nutritional supplementation with choline during the perinatal period. Maternal choline supplementation during lactation, modestly increases locomotor activity levels and improves motor coordination in Mecp2^{1lox} mutant male offspring; cognitive deficits remained unaltered (Nag & Berger-Sweeney, 2007).

Although cholinergic dysfunction is the most consistently documented neurochemical abnormality in RTT, abnormalities in biogenic amines, glutamate, substance P, and growth factors, particularly BDNF, have all been reported. It remains unclear which neurochemical changes are primary and which may be secondary. In an animal model of RTT, glutamatergic deficits preceded the cholinergic abnormalities and also preceded declines in neuronal integrity [Ward 2009 PMID: 19012748].

The Mecp2 R168X and Mecp2J mouse models of RTT are excellent animal model in which to test the efficacy of galantamine n-butylcarbamate in Rett Syndrome. R168X is the most common point mutation in humans, and the Mecp2J mouse model is a functional deletion mutation. These mouse models exhibit phenotypes that are highly reminiscent of the human condition [Stearns 2007 PMID: 17383101]. Using both male and female mutant mice on a battery of behavioral and physiological tasks, as described previously [in Stearns 2007 PMID: 17383101]will allow us assess the preclinical potential of this compound, including an assessment of the efficacy of galantamine n-butylcarbamate in acute and chronic dosing schemes to improve the RTT-related phenotype. Suggested guidelines for preclinical trials in RTT [Katz 2012 PMID: 23115203]outline the methodologies, protocols, and rigorous standards to ensure that preclinical experiments in mouse models of RTF are likely to translate into clinical success.

Galantamine has the structure:

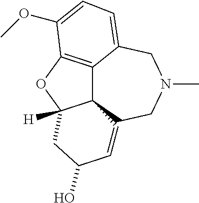

Galantamine is approved for the treatment of patients with mild to moderate Alzheimer's disease. It is administered in a dose of from 16mg to 24 mg/day.

U.S. Pat. No. 4,663,318, describes the use of galantamine, a known cholinesterase inhibitor, in the treatment of Alzheimer's disease. PCT publication WO 8808708, describes the use of analogs of galantamine and lycoramine for a similar purpose. U.S. Pat. No. 6,670,356, describes the effects of analogs of galantamine and lycoramine in modulation of nicotinic receptors and in treating and retarding the progression of Alzheimer's and Parkinson's diseases, neuroprotection against neurodegenerative disorders. At the time of these patents, Alzheimer's disease understood to be a condition that manifested itself by dementia and its underlying causes were only beginning to be understood. The treatments described in these earlier patents addressed factors involved in such dementia, namely reducing the activity of acetylcholinesterase so as to limit the reduction in availability of the neurotransmitter acetylcholine that arises from the action of acetylcholinesterase thereon and indirect stimulation of nicotinic receptors by allosteric modulation thereof to improve their functioning.

### SUMMARY OF THE INVENTION

From the first aspect the present invention provides a method treatment of patients with Rett syndrome which comprises administering thereto a therapeutically acceptable dose of a galantamine analog wherein the hydroxy group is replaced by a carbamate, carbonate or ester group and the methoxy group may be replaced by another alkoxy group of from two to six carbon atoms, a hydroxy group, hydrogen, an alkanoyloxy group or 2 to 10 carbon atoms, a benzoyloxy or substituted benzoyloxy group, a carbonate group of 1 to 10 carbon atoms or a carbamate group such as a mono alkyl or dialkyl or an aryl carbamate wherein the alkyl groups or aryl groups contain from 1 to 10 carbons; and the N-methyl group may be replaced by hydrogen, alkyl of 1 to 10 carbon atoms, benzyl, cyclopropylmethyl group or a substituted or unsubstituted benzoyloxy group.

Typically the group used to replace the hydroxyl group will be an alkanoyloxy group or 2 to 10 carbon atoms, a benzoyloxy or substituted benzoyloxy group, a carbonate group of 1 to 10 carbon atoms or a carbamate group such as a mono alkyl or dialkyl or an aryl carbamate wherein the alkyl groups or aryl groups contain from 1 to 10 carbons. Ester and carbamate groups are particularly useful. Commonly, the methoxy and methyl groups of galantamine will be left unchanged. Mono alkyl carbamates of 2 to 8 carbon atoms may be particularly useful.

One particularly useful compound is the n-butylcarbamate derivative of galantamine, having the structure:

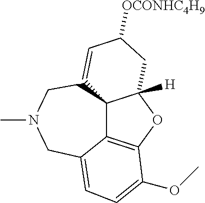

The IC₅₀ for galantane n-butylcarbamate is 10.9×10⁻⁷ M as compared to 3.97×10⁻⁷ M for galantamine.

This compound was first described in Han et al as a cholinesterase inhibitor in Bioorg. & Medicinal Chemistry Letters 1, 11 579-580 (1991).

The butylcarbamate differed from galantamine in adverse effects. (Han et al, Eur J Med Chem 1992, 27, 673) Decreased motility which appeared at 5 mg/kg in galantamine-treated animals was not observed up to30 mg/kg of the analog. At doses of 50-100 mg/kg of the n-butylcarbamate, mice were wobbly and off-balance with rapid heart rate still present at 4 hours, but were recovered at 24 hours. There was no lethality up to 100 mg/kg. The LD50 of galantamine is 10 mg/kg. Mice injected IP with 10, 15 and 20 mg/kg galantamine develop seizures at an average of 8, 6 and 4 minutes respectively (Fonck et al, J Neurosci 2003, 23, 7, 2582).

Galantamine n-butylcarbarmate is predicted to have 80% oral bioavailability, based on in vitro permeability of a layer of CaCo-2 cells, derived from a human colorectal carcinoma, as shown below.

| | | Assay | mean A->B | |
|---|---|---|---|---|
| | test conc | duration | Pₐₚₚ^{a} | |
| Client ID | (μM) | (hr) | (10⁻⁶ cm s⁻¹) | comment |
| Ranitidine | 50 | 2 | 1.1 | low permeability |
| | | | | control |
| Warfarin | 50 | 2 | 34.7 | high permeability |
| | | | | control |
| Galanthamine | 50 | 2 | 20.8 | |
| Carbamate | | | | |

^{a}Apparent permeability

In an in-vitro preparation of liver microsomes, the half-life of galantamine n-butylcarbamate was greater than 60 minutes.

Galantamine n-butylcarbamate, based on animal and in-vitro studies, appears to be well tolerated, safe, orally bioavailable, stable in plasma, and effective in enhancing learning at lower doses than galantamine. It enhances neuronal electrophysiological activity via the galantamine positive allosteric modulatory site on nicotinic receptors.

Diagnosis of Patients For Treatment by the Present Invention May be Effected by Clinical Examination and Genetic Testing.

RTT is invariably associated with a genetic defect in the MEM gene and treatment may be useful for patients who have been determined to have such a mutation even if no clinical symptoms are displayed.

Compositions suitable for use in treatments according to the invention are typically suitable for oral administration such as tablets, capsules, or lozenges containing from 0.1 to 40 mg. of the active compound depending upon the activity and half-life of the compound. Compositions using the butylcarbamate will typically contain, for example in the range 1 to 10 mg, or 2 to 25 mg, or 5 to 40 mg per dose.

Oral dosage forms may be sustained dosage formulations in which the particles of the active compound are coated so as to delay release into the blood stream for example by coating with a pharmaceutically acceptable polymer that is dissolved in gastric juices such as polyvinyl pyrrolidone and then sizing the particles and incorporating specific ratios of particles of particular sizes into a tablet, capsule or lozenge so that particles having different degrees of thickness of coating are released at different times. In the present case, the coating technique will desirably result in most of the active compound being released within twelve hours of administration. Alternative means of application may include for example transdermal patches in which case the objective is to provide administration of a dosage at a rate of . . . to 0.01 to 10 mg per hour.

Other dosage forms may be used if desired. For example nasal or parenteral including dosage formulations to assist passage of the blood-brain barrier.

For the purpose of nasal or parenteral therapeutic administration, the active compounds of the invention may be incorporated into a solution or suspension. These preparations typically contain at least 0.1% of active compound, for example between 0.5 and about 30% of the weight thereof. Preferred compositions and preparations according to the present inventions are prepared so that a nasal or parenteral dosage unit contains between 0.1 to 10 milligrams of active compound.

The solutions or suspensions may also include the following components: a sterile diluent such as water for infection, saline solution, fixed oils, polyethylene glycols, glycerine, propylene glycol or other synthetic solvents; antibacterial agents, such as benzyl alcohol or methyl parabens; antioxidants such as ascorbic acid or sodium bisulfite; chelating agents such as ethylene-diamine tetraacetic acid; buffers such as acetates; citrates or phosphates and agents for the adjustment of tonicity such as sodium chloride or dextrose. Parenteral multiple dose vials may be of glass or plastic.

Typical dosage rates in administration of the active ingredients depend on the nature of the compound that is used and in intravenous administration are in the range of 0.01 to 2.0 mg per day and per kilogram of body weight based on the physical condition and other medications of the patient.

Liquid formulations for nasal or intra-cerebroventricular administration at a concentration of 0.1 to 5 mg of active ingredient/ml. The compounds according to the invention can also be administered by a transdermal system, in which 0.1 to 10 mg/day is released. A transdermal dosage system may consist of a storage layer that contains 0.1 to 30 mg of the active substance as a free base of salt, in case together with a penetration accelerator, e.g., dimenyl sulfoxide, or a carboxylic acid, e.g., octanoic acid, and a realistic-looking polyacrylate, e.g., hexylacrylate/vinyl acetate/acrylic acid copolymer including softeners, e.g., isopropylmyristate. As a covering, an active ingredient-impermeable outside layer, e.g., a metal-coated, siliconized polyethylene patch with a thickness of for example, 0.35 mm, can be used. To produce an adhesive layer, e.g., a dimethytarnino-methacrylaternethacrylate copolymer in an organic solvent can be used.

The determination of a particular dose for any given patient will be a matter for the judgment of the physician treating the patient. However, suitable dosages may be determined by starting with a low dose and increasing if there is insufficient response.

As noted above, these dosages may be considerably lower than the typical 0.2 to 100 mg, such as 0.2 to 10 mg, or 1 to 50 mg.

Cognitive deficits are particularly resistant to amelioration in RTT and in animal models of RYE Given galantamine butyl carbamate's ability to enhance cognitive performance in mice, and its potential to stimulate nicotinic receptors, which are reduced in RTT [Yasui 2011], it is likely to ameliorate the cognitive deficits in this disorder. Potentiation of nicotinic receptors facilitates release of a number of different neurotransmitters including dopamine, glutamate, and GABA. Given that dopaminergic neurotransmission is decreased in RTT and that abnormal functioning of MeCP2 in GABAergic neurons alone recapitulates most RTT symptoms, administration of galantamine butyl carbamate has the potential to improve the clinical outcome of RTT girls by enhancing synaptic function in cholinergic, dopaminergic, and GABAergic pathways.

Potentiation of nicotinic receptors facilitates release of a number of different neurotransmitters including dopamine, glutamate, and GABA. Given that dopaminergic neurotransmission is decreased in RTT and that abnormal functioning of MeCP2 in GABAergic neurons alone recapitulates most RTT symptoms, administration of galantamine butyl carbamate has the potential to improve the clinical outcome of RTT girls by enhancing synaptic function in cholinergic, dopaminergic, and GABAergic pathways.

Compositions suitable for use in treatments according to the invention are typically suitable for oral administration such as tablets, capsules, or lozenges containing from 0.1 to 40 mg. of the active compound depending upon the activity and half-life of the compound. Compositions using the butylcarbamate will typically contain, for example in the range 1 to 10 mg, or 2 to 25 mg, or 5 to 40 mg per dose.

Oral dosage forms may be sustained dosage formulations in which the particles of the active compound are coated so as to delay release into the blood stream for example by coating with a pharmaceutically acceptable polymer that is dissolved in gastric juices such as polyvinyl pyrrolidone and then sizing the particles and incorporating specific ratios of particles of particular sizes into a tablet, capsule or lozenge so that particles having different degrees of thickness of coating are released at different times. In the present case, the coating technique will desirably result in most of the active compound being released within twelve hours of administration. Alternative means of application may include for example transdermal patches in which case the objective is to provide administration of a dosage at a rate of 0.01 to 10 mg per hour.

Other dosage forms may be used if desired. For example nasal or parenteral including dosage formulations to assist passage of the blood-brain barrier.

For the purpose of nasal or parenteral therapeutic administration, the active compounds of the invention may be incorporated into a solution or suspension. These preparations typically contain at least 0.1% of active compound, for example between 0.5 and about 30% of the weight thereof. Preferred compositions and preparations according to the present inventions are prepared so that a nasal or parenteral dosage unit contains between 0.1 to 10 milligrams of active compound.

The solutions or suspensions may also include the following components: a sterile diluent such as water for injection, saline solution, fixed oils, polyethylene glycols, glycerine, propylene glycol or other synthetic solvents; antibacterial agents, such as benzyl alcohol or methyl parabens; antioxidants such as ascorbic acid or sodium bisulfite; chelating agents such as ethylene-diamine tetraacetic acid; buffers such as acetates; citrates or phosphates and agents for the adjustment of tonicity such as sodium chloride or dextrose. Parenteral multiple dose vials may be of glass or plastic.

Typical dosage rates in administration of the active ingredients depend on the nature of the compound that is used and in intravenous administration are in the range of 0.01 to 2.0 mg per day and per kilogram of body weight based on the physical condition and other medications of the patient.

Liquid formulations for nasal or intra-cerebroventricular administration at a concentration of 0.1 to 5 mg of active ingredient/ml. The compounds according to the invention can also be administered by a transdermal system, in which 0.1 to 10 mg/day is released. A transdermal dosage system may consist of a storage layer that contains 0.1 to 30 mg of the active substance as a free base of salt, in case together with a penetration accelerator, e.g., dimenyl sulfoxide, or a carboxylic acid, e.g., octanoic acid, and a realistic-looking polyacrylate, e.g., hexylacrylate/vinyl acetate/acrylic acid copolymer including softeners, e.g., isopropylmyristate. As a covering, an active ingredient-impermeable outside layer, e.g., a metal-coated, siliconized polyethylene patch with a thickness of, for example, 0.35 mm, can be used. To produce an adhesive layer, e.g., a dimethylamino-methacrylate/methacrylate copolymer in an organic solvent can be used.

The determination of a particular dose for any given patient will be a matter for the judgment of the physician treating the patient. However, suitable dosages may be determined by starting with a low dose and increasing if there is insufficient response. As noted above, these dosages may be considerably lower than the typical 0.2 to 100 mg, such as 0.2 to 10 mg, or 1 to 50 mg with appropriate adjustment for body weight if the patient is not an adult.

The Mecp2 R168X and Mecp2J mouse models of RTT are excellent animal model in which to test the efficacy of galantamine n-butylcarbamate in Rett Syndrome. R168X is the most common point mutation in humans, and the Mecp2J mouse model is a deletion mutation. These mouse models exhibit phenotypes that are highly reminiscent of the human condition [Stearns 2007 PMID: 17383101]. Using both male and female mutant mice on a battery of behavioral and physiological tasks, as described previously [in Stearns 2007 PMID: 17383101] will allow us assess the preclinical potential of this compound, including an assessment of the efficacy of galantamine n-butylcarbamate in acute and chronic dosing schemes to improve the RTT-related phenotype. Suggested guidelines for preclinical trials in RTT [Katz 2012 PMID: 23115203] outline the methodologies, protocols, and rigorous standards to ensure that preclinical experiments in mouse models of RTT are likely to translate into clinical success.

Galantamine n-butylcarbamate affects cardiac and motor functions at very high doses (50 and 100 mg/kg). These negative side effects could be especially detrimental in WIT girls who have cardiac abnormalities and pronounced motoric impairments. Because the cholinergic system (which is modulated by galantamine n-butylcarbamate) affects both motor and respiratory functions, we monitored locomotor and respiratory functions in response to the drug treatment. In order to assess cognitive function in response to the drug treatment, we used a novel object recognition task because this task is one of the most consistent tasks in which female Mecp2 mice show significant deficits (Stearns et al. 2007 Neuroscience 146: 907-921 PMID 17383101; Katz, Berger-Sweeney et al., 2012 Disease Model Mech 5: 733-45. PMID 23115203).

Locomotor activity was monitored using methods described previously (Shaevitz et al. 2013 Genes Brain Behav. 12(7): 732-40. doi: 1111111./gbb,12070). Mecp2 mutant males (between 1 and 3 months old) and females (between 3 and 6 months old) and age-matched controls were monitored for one-hour prior to and 12 hours after drug (or vehicle: 20% DMSO in saline) administration (one set of mice received IP injections and one set of mice received oral gavage) in doses that ranged from 0.1-20 mg/kg). Activity was measured across the 12-h dark cycle using a photobeam activity system (San Diego Instruments, San Diego, Calif., USA). Mice were placed individually into a cage (47×25×21 cm) inside a rectangular arena equipped with a 3×8 array of photobeams. The average number of ambulatory (two adjacent) and fine (repeated single) beam breaks per hour over the 12 h was compared. [N=2 mice/group at each dose and each administration route; for the vehicle controls, N=6 WT/group; N=6 Mecp2/group]. Data were analyzed using repeated measures analysis of variance.

Respiratory function was monitored in a plethysmograph (EMKA Technologies) for 30 minutes prior to and 1 hour after drug administration (one set of mice received IP injections and one set of mice received oral gavage). [N=2 mice/group at each dose and each administration route; for the vehicle controls, N=6 WT/group; N=6 Mecp2/group]. Data were analyzed using repeated measures analysis of variance.

Doses of the drug that did not impair motor or respiratory functions were then considered to be safe and well tolerated.

Cognitive function was assessed using the novel object recognition (NOR) task using methods previously described (Schaevitz et al. 2013). Female mice were tested because best practice suggests that pre-clinical trials of drugs should emphasize results in female models given that RTT is most prevalent in girls (Katz, Berger-Sweeney et al., 2012 Disease Model Mech. 5:733-45. PMD: 23115203). Novel object memory was assessed. during three sessions. This task relies on the innate tendency of a mouse to explore unfamiliar objects vs. familiar objects. Testing was performed in an open-field arena. Twenty-four hours prior to training, mice were habituated to the arena for 10 min. Ninety minutes before training, the mice were administered drug or vehicle (0.1, 0.5, 1.0, 2.5 and 5.0 mg/kg IP). During training, mice were given 10 min to explore two identical Lego Objects (A +A). Short- and long-term object memory were assessed in two subsequent sessions (24 h after the completion of training) during which mice were given 10 min to explore the familiar (A) or a novel (B or C) object. The duration of exploration (defined as the mouse's snout or forelimbs physically touching or approaching within 1 cm of an object) of familiar and novel objects was measured. The amount of time spent exploring the novel object over the total time exploring both novel and familiar objects in each session was used to measure object memory. [N==6/dose of 0.1, 0.5 and 1.0; N=1/dose of 2.5 and 5.0 mg/kg; for vehicle N=6 WT and N=6 Mecp2 mice.] Given the small number of mice tested at each dose, we combined mice into groups of Mecp2 or controls, and vehicle or drug-treated Mecp2 mice and analyzed data the using Chi-squared analyses to determine whether there were differences amongst group of those that learned the NOR task and those that did not.

### Results

Ambulatory and fine motor movements were not significant altered at any of the doses of the drug tested. We have shown previously (Schaevitz et al. 2013) that ambulatory movements in Mecp2 males is significantly lower than in wildtype mice; in Mecp2 females were also significantly lower than wildtype, but the impairment was milder. Doses of the drug (administered either IP or by gavage between 0.1 and 20 mg/kg) did not impair locomotor activity in the Mecp2 mice of either sex. Also, the same doses and administration routes of the drug did not affect respiratory activity. Therefore, the drug was safe and well tolerated at doses between 0.1 and 20 mg/kg in Mecp2 mice of both sexes, as well as controls.

For the novel Objection recognition task data, we created a matrix of all wildtype and. Mecp2 females who were administered the vehicle and a second matrix comparing Mecp2 females with and without the drug (all doses combined). Mice were divided into two categories: those who learned the novel object task (had object recognition scores above chance level >0.5) and those that did not learn the novel object task (had object recognition scores at or below chance levels ≦0.5). We asked two questions:
1) Do the Mecp2 females (administered vehicle) perform significantly worse than WT controls on the task?

| | NOR scores = or below 0.5 | NOR scores above 0.5 |
|---|---|---|
| Mecp2 | 83% | 17% |
| WT | 33% | 67% |

The wildtype mice learned the NOR task but the Mecp2 mice did not learn the task [Chi square, (df = 1, N = 12) = 6.75, p = 0.0094].

2) Does galantamine n-butylcarbamate improve performance in the Mecp2 mice on the task?

| | NOR scores = or below 0.5 | NOR scores above 0.5 |
|---|---|---|
| Mecp2 | 83% | 17% |
| (Vehicle) | | |
| Mecp2 | 65% | 35% |
| (drugs) | | |

The Mecp2 mice treated with galantamine n-butylcarbamate learned the NOR task significantly better than vehicle-injected Mecp2 mice [Chi square, (df = 1, N = 12) = 4.592, p = 0.0321].

Therefore, our data show that galantamine n-butylcarbamate improves memory for a novel object and cognitive performance in a female mouse model of RTT syndrome at doses that do not impair locomotor activity or respiratory functions.

1. A method for treating patients with Rett Syndrome which comprises administering to a patient in need thereof a therapeutically effective dose of a galanthamine analog wherein
the hydroxy group is replaced by a carbamate, carbonate or ester group the methoxy group is optionally replaced by another alkoxy group of from two to six carbon atoms, a hydroxy group, hydrogen, an alkanoyloxy group or 2 to 10 carbon atoms, a benzoyloxy or substituted benzoyloxy group, a carbonate group of 1 to 10 carbon atoms or a carbamate group such as a mono alkyl or dialkyl or an aryl carbamate wherein the alkyl groups or aryl groups contain from 1 to 10 carbons; and
the N-methyl group is optionally replaced by hydrogen, alkyl of 1 to 10 carbon atoms, benzyl, cyclopropylmelhyl group or a substituted or unsubstituted benzoyloxy group.

2. A method as claimed in claim 1, wherein the hydroxyl group of galantamine is replaced by an alkanoyloxy group or 2 to 10 carbon atoms, a benzoyloxy or substituted benzoyloxy group, a carbonate group of 1 to 10 carbon atoms or a carbamate group such as a mono alkyl or dialkyl or an aryl carbamate wherein the alkyl groups or aryl groups contain from 1 to 10 carbons.

3. A method as claimed in claim 1, wherein the hydroxy group of galantamine is replaced by a mono alkyl carbamate group of 2 to 8 carbon atoms.

4. A method as claimed in claim 3, wherein the hydroxy group of galantamine is replaced by an n-butyl carbamate group.

5. A method as claimed in claim 1, wherein the methoxy and methyl groups of galantamine are unchanged.

6. A method as claimed in claim 4, wherein the methoxy and methyl groups of galantamine are unchanged.

7. A method as claimed in claim 1, wherein the dose of a galantamine analog is from 0.2 to 100 mg.

8. A method as claimed in claim 4 wherein n-butyl carbamate is administered in dosages of 1 to 10 mg, or 2 to 25 mg, or 5 to 40 mg per dose.

9. A method as claimed in claim 1, wherein the galantamine analog is administered as an oral dosage form in which the particles of the galaniamine analogre coated so as to delay release into the blood stream by coating with a pharmaceutically acceptable polymer that is dissolved in gastric juices.
